(19) United States
(12) Patent Application Publication
(10) Pub. No.: US 2021/0068674 A1
(43) Pub. Date: Mar. 11, 2021

(54) TRACK USER MOVEMENTS AND BIOLOGICAL RESPONSES IN GENERATING INPUTS FOR COMPUTER SYSTEMS

(71) Applicant: Finch Technologies Ltd., Fish Bay (VG)

(72) Inventors: Viktor Vladimirovich Erivantcev, Ufa (RU); Alexey Ivanovich Kartashov, Moscow (RU); Iakov Evgenevich Sergeev, Ufa (RU); Ravil Salavatovich Shafikov, Kumertau (RU); Gary Stuart Yamamoto, Sacramento, CA (US)

(21) Appl. No.: 17/008,219

(22) Filed: Aug. 31, 2020

Related U.S. Application Data
(60) Provisional application No. 62/897,131, filed on Sep. 6, 2019.
(60) Provisional application No. 62/933,818, filed on Nov. 11, 2019.

(51) Int. Cl. A61B 5/0205; A61B 5/11; A61B 5/00; G06F 3/01

(52) U.S. Cl. CPC A61B 5/02055; A61B 5/1112; A61B 5/746; A61B 5/742; A61B 5/1114; A61B 2562/029; G06F 3/011; A61B 2562/0219; A61B 2562/028; A61B 2562/0247; A61B 5/6824

(57) ABSTRACT

A computing system having a plurality of sensor modules configured to be attached to a user. At least a first subset of the sensor modules each has an inertial measurement unit; and at least a second subset of the sensor modules each has one or more biological response sensors. The computing system further includes a computing device in communication with the sensor modules. The computing device is configured to control an application based on motion data of the user measured by inertial measurement units in the sensor modules and biological responses determined by sensor modules.

### RELATED APPLICATIONS

The present application claims the benefit of priority to U.S. Provisional App. Ser. No. 62/897,131, filed on Sep. 6, 2019, the entire disclosure of which application is hereby incorporated herein by reference. The present application claims the benefit of priority to U.S. Provisional App. Ser. No. 62/933,818, filed on Nov. 11, 2019, the entire disclosure of which application is hereby incorporated by reference.

The present application relates to Prov. U.S. Pat. App. Ser. No. 62/897,131, filed Sep. 6, 2019 and entitled “Track User Movements and Biological Responses in Generating Inputs for Computer Systems”, and U.S. patent application Ser. No. 15/787,555, filed Oct. 18, 2017, issued as U.S. Pat. No. 10,379,613 on Aug. 13, 2019, and entitled “Tracking Arm Movements to Generate Inputs for Computer Systems,” the entire disclosure of which application is hereby incorporated herein by reference.

The present application further relates to U.S. patent application Ser. No. 15/492,915, filed Apr. 20, 2017, published as U.S. Pat. App. Pub. No. 2017/0308165 on Oct. 26, 2017, and entitled “Devices for Controlling Computers based on Motions and Positions of Hands,” U.S. patent application Ser. No. 15/817,646, filed Nov. 20, 2017, published as U.S. Pat. App. Pub. No. 2018/0313867 on Nov. 1, 2018, and entitled “Calibration of Inertial Measurement Units Attached to Arms of a User to Generate Inputs for Computer Systems,” U.S. patent application Ser. No. 15/813,813, filed Nov. 15, 2017, published as U.S. Pat. App. Pub. No. 2018/0335834 on Nov. 22, 2018, and entitled “Tracking Torso Orientation to Generate Inputs for Computer Systems,” U.S. patent application Ser. No. 15/847,669, filed Dec. 19, 2017, published as U.S. Pat. App. Pub. No. 2019/0187784 on Jun. 20, 2019, and entitled “Calibration of Inertial Measurement Units Attached to Arms of a User and to a Head Mounted Device,” U.S. patent application Ser. No. 15/868,745, filed Jan. 11, 2018, published as U.S. Pat. App. Pub. No. 2019/0212359 on Jul. 11, 2019, and entitled “Correction of Accumulated Errors in Inertial Measurement Units Attached to a User,” U.S. patent application Ser. No. 15/864,860, filed Jan. 8, 2018, published as U.S. Pat. App. Pub. No. 2019/0212807 on Jul. 11, 2019, and entitled “Tracking Torso Leaning to Generate Inputs for Computer Systems,” U.S. patent application Ser. No. 15/973,137, filed May 7, 2018, and entitled “Tracking User Movements to Control a Skeleton Model in a Computer System,” U.S. patent application Ser. No. 15/996,389, filed Jun. 1, 2018, issued as U.S. Pat. No. 10,416,755 on Sep. 17, 2019, and entitled “Motion Predictions of Overlapping Kinematic Chains of a Skeleton Model used to Control a Computer System,” U.S. patent application Ser. No. 16/044,984, filed Jul. 25, 2018, and entitled “Calibration of Measurement Units in Alignment with a Skeleton Model to Control a Computer System,” U.S. patent application Ser. No. 16/375,108, filed Apr. 4, 2019, and entitled “Kinematic Chain Motion Predictions using Results from Multiple Approaches Combined via an Artificial Neural Network,” U.S. patent application Ser. No. 16/534,674, filed Aug. 7, 2019, and entitled “Calibration of Multiple Sensor Modules Related to an Orientation of a User of the Sensor Modules,” the entire disclosures of which applications are hereby incorporated herein by reference.

### FIELD OF THE TECHNOLOGY

The embodiments disclosed herein relate to computer input devices in general and more particularly but not limited to input devices for virtual reality and/or augmented/mixed reality applications implemented using computing devices, such as mobile phones, smart watches, similar mobile devices, and/or other devices.

### BACKGROUND

U.S. Pat. App. Pub. No. 2014/0028547 discloses a user control device having a combined inertial sensor to detect the movements of the device for pointing and selecting within a real or virtual three-dimensional space.

U.S. Pat. App. Pub. No. 2015/0277559 discloses a finger-ring-mounted touchscreen having a wireless transceiver that wirelessly transmits commands generated from events on the touchscreen.

U.S. Pat. App. Pub. No. 2015/0358543 discloses a motion capture device that has a plurality of inertial measurement units to measure the motion parameters of fingers and a palm of a user.

U.S. Pat. App. Pub. No. 2007/0050597 discloses a game controller having an acceleration sensor and a gyro sensor. U.S. Pat. No. D772,986 discloses the ornamental design for a wireless game controller.

Chinese Pat. App. Pub. No. 103226398 discloses data gloves that use micro-inertial sensor network technologies, where each micro-inertial sensor is an attitude and heading reference system, having a tri-axial micro-electromechanical system (MEMS) micro-gyroscope, a tri-axial micro-acceleration sensor and a tri-axial geomagnetic sensor which are packaged in a circuit board. U.S. Pat. App. Pub. No. 2014/0313022 and U.S. Pat. App. Pub. No. 2012/0025945 disclose other data gloves.

The disclosures of the above discussed patent documents are hereby incorporated herein by reference.

### SUMMARY

A system is disclosed. The system includes a plurality of sensor modules configured to be attached to a user. At least a first of the plurality of sensor modules have an inertial measurement unit. At least a second of the plurality of sensor modules have one or more biological response sensors having a biological sensor module. A computing device is in communication with at least one of the plurality of sensor modules. The computing device is configured to control an application based on motion data of the user measured by inertial measurement units in the plurality of sensor modules and biological responses determined by the biological sensor module.

A method is also disclosed. The method includes receiving, by a computing device, motion data from an arm module identifying an orientation of an upper arm of a user. The method further includes receiving, by the computing device, motion data from a handheld module identifying an orientation of a hand of the user. The method further includes receiving, by the computing device, biological responses from a biological sensor module. The method includes calculating, by the computing device, an orientation of a forearm of the user connecting the hand and the upper arm, wherein the orientation is calculated without a sensor module being present on the forearm. The method further includes controlling an application based on the calculated orientation of the forearm of the user, the received motion data from the arm module, the received motion data from the handheld module, and the biological responses.

### BRIEF DESCRIPTION OF THE DRAWINGS

The embodiments are illustrated by way of example and not limitation in the figures of the accompanying drawings in which like references indicate similar elements.

FIG. 1 illustrates a system to track arm movements according to one embodiment.

FIG. 2 illustrates a system to control computer operations according to one embodiment.

FIG. 3 illustrates a skeleton model of an arm.

FIG. 4 illustrates the determination of the orientation of a forearm according to one embodiment.

FIG. 5 shows a method to compute the orientation of a forearm according to one embodiment.

FIG. 6 shows a detailed method to compute the orientation of a forearm according to one embodiment.

FIG. 7 shows a sensor module sticking to an upper arm according to one embodiment.

FIG. 8 shows a sensor module having a sticky pad for attaching to a user according to one embodiment.

FIG. 9 shows a sensor module having biological response sensors and an inertial measurement unit according to one embodiment.

FIG. 10 shows another sensor module having biological response sensors, an inertial measurement unit, and audio input/output devices according to one embodiment.

### DETAILED DESCRIPTION

The following description and drawings are illustrative and are not to be construed as limiting. Numerous specific details are described to provide a thorough understanding. However, in certain instances, well known or conventional details are not described to avoid obscuring the description. References to one or an embodiment in the present disclosure are not necessarily references to the same embodiment; and, such references mean at least one.

At least some embodiments disclosed herein allow arm movement tracking without a sensor device attached to the forearm. The forearm orientation is estimated, predicted, or computed from the orientation of the upper arm connected to the forearm and the orientation of the hand connected to the forearm.

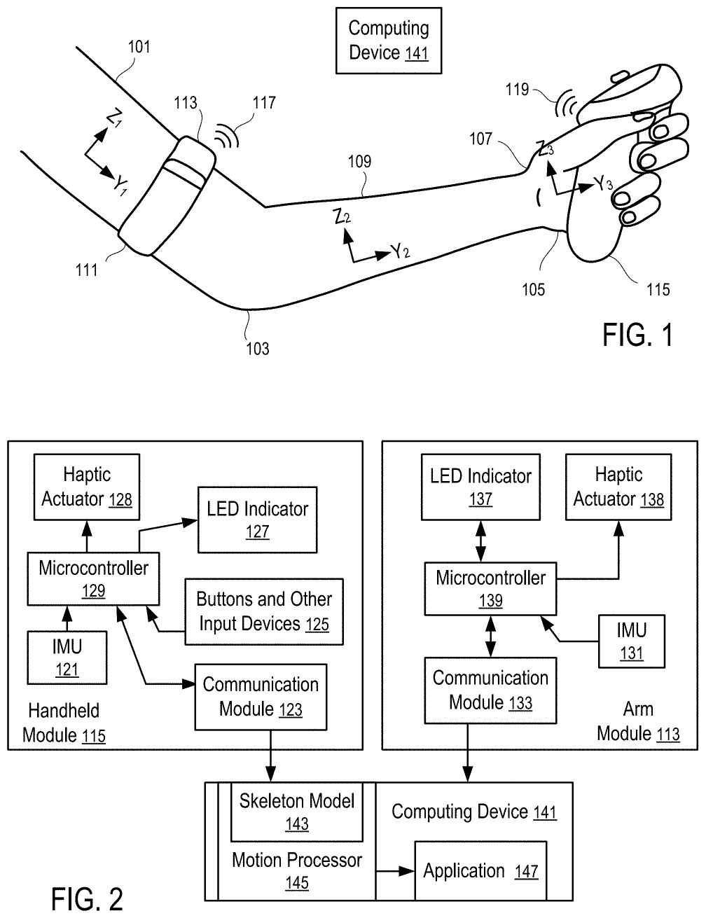

FIG. 1 illustrates a system to track arm movements according to one embodiment.

In FIG. 1, an elbow joint (103) of a user connects an upper arm (101) and a forearm (109) of the user; and a wrist (107) connects the forearm (109) to a hand (105) of the user.

The orientation of the upper arm (101) is tracked/determined using an arm module (113) that is attached to the upper arm (101) via an armband (111). The orientation of the upper arm (101) is represented by a local coordinate system X₁Y₁Z₁, where the lengthwise direction Y₁ is in parallel with the direction from the shoulder to the elbow joint (103), the direction X₁ is in parallel with the direction from the inner side of the upper arm (101) to the outer side of the upper arm (101), and the direction Z₁ is in parallel with the direction from the back side of the upper arm (101) to the front side of the upper arm (101).

The orientation of the hand (105) is tracked/determined using a handheld module (115). The orientation of the hand (105) is represented by a local coordinate system X₃Y₃Z₃, where the lengthwise direction Y₃ is in parallel with the direction from the wrist (105) to the fingers, the direction X₃ is in parallel with the direction from the back of the hand (105) to the palm of the hand (105), and the direction Z₃ is in parallel with the direction from the edge of the palm to the thumb on the hand (105).

Preferably, the arm module (113) and the handheld module (115) separately report their motion/orientation parameters to a computing device (141) using wireless connections (117 and 119), such as a personal area wireless network connection (e.g., Bluetooth connections), or a local area wireless network connection (e.g., Wi-Fi connections).

Alternatively, the arm module (113) may report its measurements to the handheld module (115) (via a wired or wireless connection); and the handheld module (115) communicates the motion/orientation measurements to the computing device (141) (e.g., via a wired or wireless connection).

For example, the handheld module (115) and the arm module (113) can be respectively a base unit (or a game controller) and an arm/shoulder module discussed in U.S. patent application Pub. Ser. No. 15/492,915, filed Apr. 20, 2017 and entitled “Devices for Controlling Computers based on Motions and Positions of Hands”, the entire disclosure of which application is hereby incorporated herein by reference.

At least some embodiments disclosed herein allow the orientation of the forearm (109) to be estimated, predicted, or calculated from the orientation of the hand (105) and the orientation of the upper arm (101) without the need for an additional sensor module to track the orientation of the forearm (109), as further discussed below.

FIG. 2 illustrates a system to control computer operations according to one embodiment. For example, the system of FIG. 2 can be implemented via attaching the handheld module (115) and the arm module (113) to the hand (105) and the upper arm (101) respectively in a way illustrated in FIG. 1.

In FIG. 2, the handheld module (115) and the arm module (113) have micro-electromechanical system (MEMS) inertial measurement units (IMUs) (121 and 131) that measure motion parameters and determine orientations of the hand (105) and the upper arm (101).

Each of the IMUs (131, 121) has a collection of sensor components that enable the determination of the movement, position and/or orientation of the respective IMU along a number of axes. Examples of the components are: a MEMS accelerometer that measures the projection of acceleration (the difference between the true acceleration of an object and the gravitational acceleration); a MEMS gyroscope that measures angular velocities; and a magnetometer that measures the magnitude and direction of a magnetic field at a certain point in space. In some embodiments, the IMUs use a combination of sensors in three and two axes (e.g., without a magnetometer).

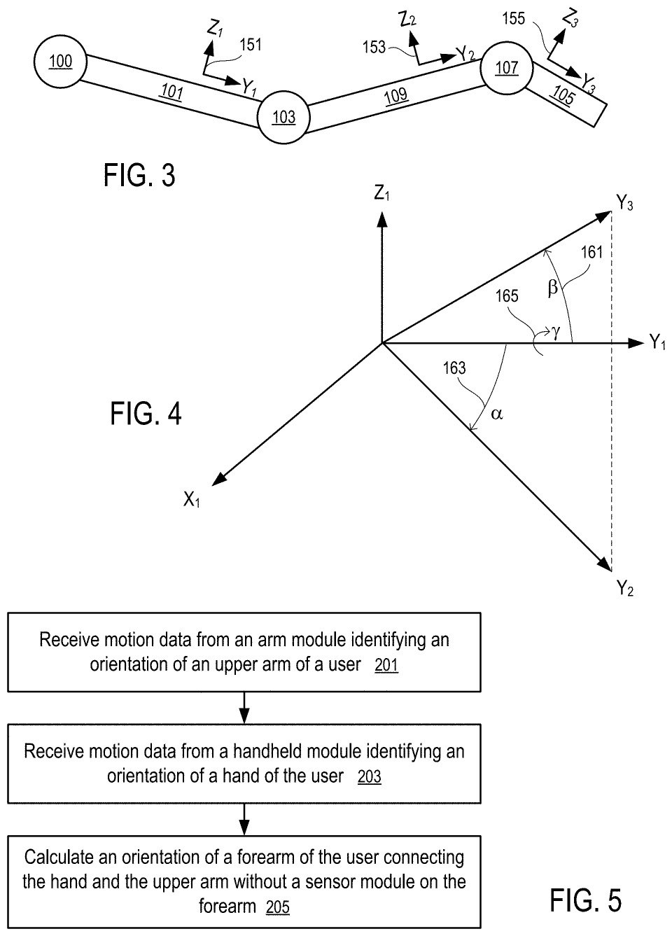

The computing device (141) has a motion processor (145), which includes a skeleton model (143) of the upper arm (101), the forearm (109), and the hand (105) connected via the elbow joint (103) and the wrist (107) (e.g., illustrated FIG. 3). The motion processor (145) controls the movements of the corresponding parts of the skeleton model (143) according to the movements/orientations of the upper arm (101) and the hand (105) measured by the arm module (113) and the handheld module (115).

Since the forearm (109) does not have an attached sensor module, the movements/orientations of the forearm (109) is calculated/estimated/predicted from the orientation of the arm module (113) and the orientation of the handheld module (115), as discussed further below.

The skeleton model (143) as controlled by the motion processor (145) generates inputs for an application (147) running in the computing device (141). For example, the skeleton model (143) can be used to control the movement of an avatar/model of the arm of the user of the computing device (141) in a video game, a virtual reality, a mixed reality, or augmented reality, etc.

In some applications, the handheld module (115) can be replaced with an arm module (113) attached to the hand (105) via holding or via a strap.

Preferably, the arm module (113) has a microcontroller (139) to process the sensor signals from the IMU (131) of the arm module (113) and a communication module (133) to transmit the motion/orientation parameters of the arm module (113) to the computing device (141). Similarly, the handheld module (115) has a microcontroller (129) to process the sensor signals from the IMU (121) of the handheld module (115) and a communication module (123) to transmit the motion/orientation parameters of the handheld module (115) to the computing device (141).

Optionally, the arm module (113) and the handheld module (115) have LED indicators (137 and 127) respectively to indicate the operating status of the modules (113 and 115).

Optionally, the arm module (113) and the handheld module (115) have haptic actuators (138 and 128) respectively to provide haptic feedback to the user via the modules (113 and 115).

Optionally, the handheld module (115) has buttons and other input devices (125), such as a touch sensor, a joystick, etc.

Typically, an IMU (e.g., 131 or 121) in a module (e.g., 113 or 115) generates acceleration data from accelerometers, angular velocity data from gyrometers/gyroscopes, and/or orientation data from magnetometers. The microcontrollers (139 and 129) perform preprocessing tasks, such as filtering the sensor data (e.g., blocking sensors that are not used in a specific application), applying calibration data (e.g., to correct the average accumulated error computed by the computing device (141)), transforming motion/position/orientation data in three axes into a quaternion, and packaging the preprocessed results into data packets (e.g., using a data compression technique) for transmitting to the host computing device (141) with a reduced bandwidth requirement and/or communication time.

Each of the microcontrollers (129, 139) may include a memory storing instructions controlling the operation of the respective microcontroller (129 or 139) to perform primary processing of the sensor data from the IMU (121, 131) and control the operations of the communication module (123, 133), and/or other components, such as the LED indicator (127, 137), the haptic actuator (128, 138), buttons and other input devices (125).

The computing device (141) may include one or more microprocessors and a memory storing instructions to implement the motion processor (145). The motion processor (145) may also be implemented via hardware, such as Application-Specific Integrated Circuit (ASIC) or Field-Programmable Gate Array (FPGA).

In some instances, one of the modules (113 and 115) is configured as a primary input device; and the other module is configured as a secondary input device that is connected to the computing device (141) via the primary input device. A secondary input device may use the microprocessor of its connected primary input device to perform some of the preprocessing tasks. A module that communicates directly to the computing device (141) is considered a primary input device, even when the module does not have a secondary input device that is connected to the computing device via the primary input device.

In some instances, the computing device (141) specifies the types of input data requested, and the conditions and/or frequency of the input data; and the modules (113 and 115) report the requested input data under the conditions and/or according to the frequency specified by the computing device (141). Different reporting frequencies can be specified for different types of input data (e.g., accelerometer measurements, gyroscope/gyrometer measurements, magnetometer measurements, position, orientation, velocity).

In general, the computing device (141) may be a data processing system, such as a mobile phone, a desktop computer, a laptop computer, a head mount virtual reality display, a personal medial player, a tablet computer, etc.

FIG. 3 illustrates a skeleton model of an arm. For example, the skeleton model of FIG. 3 can be used in the motion processor (145) of FIG. 2 to determine the orientation of the forearm (109) that does not have an attached sensor module, as illustrated in FIG. 1.

FIG. 3 shows the geometrical representations of the upper arm (101), the forearm (109), and the hand (105) in relation with the elbow joint (103) and the wrist (107) relative to the shoulder (100).

Each of the upper arm (101), the forearm (109), and the hand (105) has an orientation relative to a common reference system (e.g., the shoulder (100), a room, or a location on the Earth where the user is positioned). The orientation of the upper arm (101), the forearm (109), or the hand (105) can be indicated by a local coordinate system (151, 153, or 155) aligned with the upper arm (101), the forearm (109), or the hand (105).

The orientation of the upper arm (101) and the orientation of the hand (105), as represented by the local coordinate systems (151 and 155) can be calculated from the motion parameters measured by the IMUs in the module (113 and 105) attached to the upper arm (101) and the hand (105).

Since the forearm (109) does not have an attached IMU for the measurement of its orientation, the motion processor (145) uses a set of assumed relations between the movements of the forearm (109) and the hand (105) to calculate or estimate the orientation of the forearm (109) based on the orientation of the upper arm (101) and the orientation of the hand (105), as further discussed below.

FIG. 4 illustrates the determination of the orientation of a forearm according to one embodiment.

In FIG. 4, the coordinate system X₁Y₁Z₁ represents the orientation of the upper arm (101), where the direction Y₁ is along the lengthwise direction of the upper arm (101) pointing from the shoulder (100) to the elbow joint (103), as illustrated in FIG. 3. The directions X₁ and Z₁ are perpendicular to the direction Y₁. The direction X₁ is parallel to the direction from the back side of the upper arm (101) to the front side of the upper arm (101); and the direction X₁ is parallel to the direction from the inner side of the upper arm (101) to the outer side of the upper arm (101).

When the arm is in a vertical direction pointing downwards with the hand (105) facing the body of the user, the lengthwise directions Y₁, Y₂, and Y₃ of the upper arm (101), the forearm (109), and the hand (105) are aligned with the vertical direction pointing downwards. When in such a position, the inner sides of the forearm (109) and the upper arm (101) are closest to the body of the user; and the outer sides of the forearm (109) and the upper arm (101) are away from the body of the user; the directions Z₁, Z₂, and Z₃ of the upper arm (101), the forearm (109), and the hand (105) are aligned with a direction pointing sideways to the user; and the directions Z₁, Z₂, and Z₃ of the upper arm (101), the forearm (109), and the hand (105) are aligned with a direction pointing to the front of the user.

Thus, the plane X₁Y₁ is parallel to the direction X₁ from the back side of the upper arm (101) to the front side of the upper arm (101), parallel to the lengthwise direction Y₁ of the upper arm (101), and perpendicular to the direction Z₁ from the direction from the inner side of the upper arm (101) to the outer side if the upper arm (101). The direction Z₁ coincides with an axis of the elbow joint about which the forearm (109) can rotate to form an angle with the upper arm (101) between their lengthwise directions. When the upper arm (101) is extended in the sideways of the user and in a horizontal position, the directions X₁ and Z₁ are aligned with (in parallel with) the front direction and vertical direction respectively.

The direction Y₂ is aligned with the lengthwise direction of the forearm (109) pointing from the elbow joint (103) to the wrist (107).

The direction Y₃ is aligned with the lengthwise direction of the hand (105) pointing from the wrist (107) towards the fingers.

When the upper arm (101) is extended in the sideways of the user and in a horizontal position, the directions Y₁, Y₂, and Y₃ coincide with the horizontal direction pointing the sideways of the user.

When the hand (105) is moved to an orientation illustrated in FIG. 4, the hand (105) can be considered to have moved from the orientation of the coordinate system X₁Y₁Z₁ through rotating (165) by an angle γ along the lengthwise direction Y₁ and then rotating (161) along the shortest arc (161) such that its lengthwise direction Y₃ arrives at the direction illustrated in FIG. 4. The rotation (161) along the shortest arc (161) corresponding to a rotation of the direction Y₁ by an angle β in a plane containing both the directions Y₁ and Y₃ along an axis perpendicular to both the directions Y₁ and Y₃ (i.e., the axis is perpendicular to the plane containing both the directions Y₁ and Y₃).

The projection of the direction Y₃ in the plane)(NI is assumed to be in the direction of the lengthwise direction Y₂ of the forearm (109). The projection represents a rotation (163) of the direction Y₁ by an angle α in the plane)(NI along the direction Z₁ according to the shortest arc (163).

It is assumed that the rotation (165) of the hand (105) along its lengthwise direction is a result of the same rotation of the forearm (109) along its lengthwise direction while the forearm (109) is initially at the orientation aligned with the coordinate system X₁Y₁Z₁. Thus, when the hand (105) has an orientation illustrated in FIG. 4 relative to the orientation (X₁Y₁Z₁) of the upper arm (101), the orientation of the forearm (109) is assumed to have moved from the orientation of the coordinate system X₁Y₁Z₁ by rotating (165) along the lengthwise direction Y₁ and then rotating (163) in the plane X₁Y₁ along the direction Z₁.

Since the rotations (165, 161 and 163) can be calculated from the orientation of the hand (105) relative to the orientation of the upper arm (101) (e.g., using the orientation data measured by the IMUs of the arm module (113) and the handheld module (115)), the orientation of the forearm (109) can be calculated from the rotations (165 and 163) without measurement data from an IMU attached to the forearm (109).

After the orientations of the upper arm (101), the forearm (109) and the hand (105) are obtained, the motion processor (145) can compute the positions of the upper arm (101), the forearm (109) and the hand (105) in a three dimensional space (relative to the shoulder (100)), which allows the application (147) to present an arm of an avatar in a virtual reality, augmented reality, or mixed reality in accordance with the movement of the arm of the user. The positions of the upper arm (101), the forearm (109) and the hand (105) in a three dimensional space (relative to the shoulder (100)) can also be used to determine the gesture made by the user in the three dimensional space to control the application (147).

FIG. 5 shows a method to compute the orientation of a forearm according to one embodiment. For example, the method of FIG. 5 can be implemented in a system illustrated in FIG. 2 with an arm module (113) and a handheld module (115) attached to a hand (105) and an upper arm (101) in a way illustrated FIG. 1 and using the geometrical relations identified via FIG. 3 and FIG. 4.

In FIG. 5, a computing device (141) receives (201) motion data from an arm module (113) identifying an orientation of an upper arm (101) of a user, receives (203) motion data from a handheld module (115) identifying an orientation of a hand (105) of the user, and calculates (205) an orientation of a forearm (109) of the user connecting the hand (105) and the upper arm (101) without a sensor module on the forearm (109).

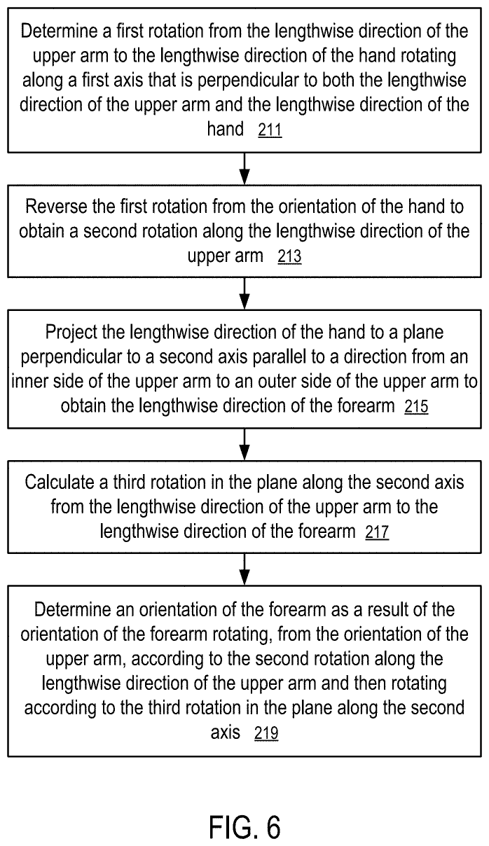

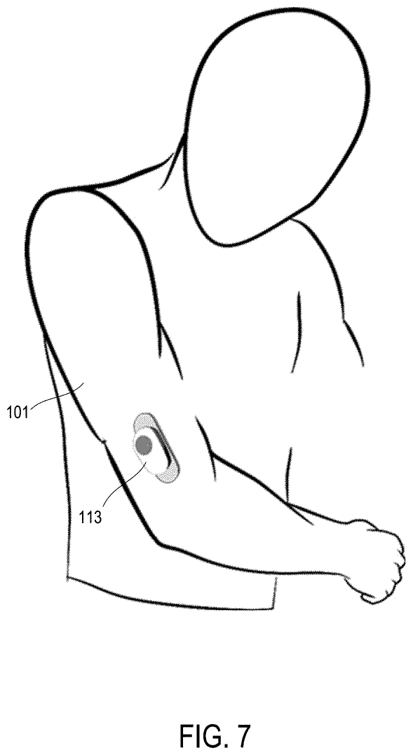

FIG. 6 shows a detailed method to compute the orientation of a forearm according to one embodiment. For example, the method of FIG. 6 can be used to implement the calculation (205) of the orientation of the forearm (109) in the method of FIG. 5.

In FIG. 6, the calculation (205) of the orientation of the forearm (109) is performed by: determining (211) a first rotation β from the lengthwise direction Y₁ of the upper arm (101) to the lengthwise direction Y₃ of the hand (105), where the first rotation rotates along a first axis that is perpendicular to both the lengthwise direction Y₁ of the upper arm (101) and the lengthwise direction Y₃ of the hand (105); reversing (213) the first rotation β from the orientation of the hand (105) to obtain a second rotation γ along the lengthwise direction Y₁ of the upper arm (101); projecting (215) the lengthwise direction Y₃ of the hand (105) to a plane X₁Y₁ that is perpendicular to a second axis Z₁ parallel to a direction from an inner side of the upper arm (101) to an outer side of the upper arm (101) to obtain the lengthwise direction Y₂ of the forearm (109); calculating (217) a third rotation α that rotates within the plane X₁Y₁ and alone the second axis Z₁ from the lengthwise direction Y₁ of the upper arm (101) to the lengthwise direction Y₂ of the forearm (109); and determining (219) an orientation of the forearm (109) as a result of the orientation X₂Y₂Z₂ of the forearm (109) rotating, from the orientation X₁Y₁Z₁ of the upper arm (101), according to the second rotation γ along the lengthwise direction Y₁ of the upper arm (101), and then rotating according to the third rotation α in the plane)(NI and along the second axis Z₁.

As illustrated in FIG. 2, each of the arm module (113) and the handheld module (115) is a sensor module that has an inertial measurement unit (IMU) (131, 121) for their orientation measurements. Preferably, the sensor module has a wireless communication device or module (133, 123) for a wireless communication link (117, 119) with the computing device (141). Alternatively, wired connections can be used. The inertial measurement unit (IMU) (131, or 121) of the sensor module (113, or 115) may include a micro-electromechanical system (MEMS) gyroscope, a magnetometer, and/or a MEMS accelerometer.

The method of FIG. 6 allows the calculation of the estimated/approximated orientation of the forearm (109) without a need for a separate inertial measurement unit or sensor module attached to the forearm (109) of the user, which arrangement reduces the cost of the system and improves user experiences.

As an example, the orientation of the forearm can be calculated using the following quaternion calculations.

The orientations of the upper arm (101) and the hand (105) can be expressed as quaternion variables qₛ and qₕ. The quaternion of rotation between the upper arm (101) and the hand (105) can be calculated as qₕₛ=qₛ⁻¹*qₕ. The lengthwise direction of the upper arm (101) is known for the orientation transformation between the upper arm (101) and the hand (105) (e.g., vector {0; 1; 0} for the left hand and vector {0; −1; 0} for the right hand). When the lengthwise direction of the upper arm (101) is expressed as vector o, the lengthwise direction of the hand (105) can be calculated as vector h=qₕₛ*o*qₕₛ⁻¹. When the projection of h in the plane containing the lengthwise direction of the upper arm and the direction from the back of the upper arm to the front of the upper arm is expressed as vector f, the quaternion of rotation (α) along the shortest arc from the vector o to the vector f can be calculated as qzx_{fs}. Similarly, the quaternion of rotation (β) along the shortest arc from the vector o to the vector h can be calculated as qzxₕₛ. Since the quaternion of rotation (γ) along the lengthwise direction of the upper arm (101) is qzxₕₛ⁻¹*qₕₛ, the quaternion of rotation between the upper arm (101) and the forearm (109) is q_{fs}=qzx_{fs} qzxₕₛ⁻¹*qₕₛ. Thus, the quaternion orientation of the forearm (109) is q_{f}=qₛ*q_{fs}.

In at least some embodiments, a sensor module (e.g., 115 or 113) is configured with a sticky surface for attaching the module to a user.

For example, an arm module (113) can have a sticky surface that can be used to attach the module (113) to the upper arm (101) of the user with or without another mechanism (e.g., an armband (111) or strap) to secure the arm module (113) to the upper arm (101).

FIG. 7 shows a sensor module (113) sticking to an upper arm (101) according to one embodiment. The sensor module (113) includes at least a micro-electromechanical system (MEMS) inertial measurement units (IMU) (131). The sensor module (113) can further include some or all of the components illustrated in FIG. 2, such as an LED indicator (137), a haptic actuator (138), a microcontroller (139), and/or a communication module (133). In an embodiment, the LED indicator (137) can include an LED display or can include one or more LED lights. Optionally, the sensor module (113) can also include one or more buttons and/or other input devices similar to the buttons and input devices (125) in a handheld module (115).

In FIG. 7, the sticky surface of the sensor module (113) has sufficient stickiness to secure the sensor module (113) to the upper arm (101) without a band or strap. The sensor module (113) sticking to the upper arm (101) does not detach from the upper arm (101) and fall off during the arm movement to control operations of a computing device (141).

Optionally, the sticky surface is configured to be attached to a side of the upper arm (101) without wrapping around the upper arm (101).

For example, the sticky surface can be created by including silicone reusable material on a side of the sensor module (113) that is configured to be attached to the bare skin of the upper arm (101), or to the clothing wore over the upper arm (101).

The lack of a band or strap allows the sensor module (113) to be attached to other parts of the user, such as the torso of the user.

Alternatively, a band or strap (111) can be used in addition to the sticky surface to attach the sensor module (113) to the upper arm (101) of the user.

By using a sticky surface to attach the sensor module (113) to the user, errors in orientation measurements generated by the IMU (131) can be reduced.

For example, when the sensor module (113) is secured to the upper arm (101) with an armband (111) but without a sticky surface, the sensor module (113) can move around the arm (101) as the armband (111) twisting around the upper arm (101). Further, the armband (111) can slip up or down the upper arm (101) as a result of stretching and relaxing of the muscles in the upper arm (101). The re-positioning of the sensor module (113) relative to the upper arm (101) can introduce errors in tracking the orientation of the upper arm (101) and the calculation of the orientation of the lower arm (101).

The sticky surface of the sensor module (113) can reduce or eliminate the relative displacement between the sensor module (113) and the upper arm (101) and thus, reduce or eliminate the errors caused by the relative displacement.

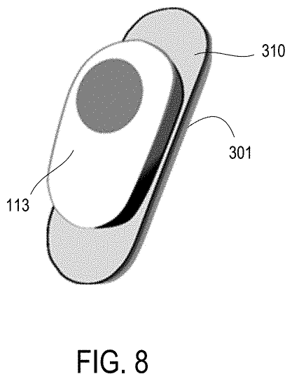

FIG. 8 shows a sensor module having a sticky pad for attaching to a user according to one embodiment.

In FIG. 8, the sensor module (113) has a housing in which the electronic components (e.g., 131, 133, 137, 138, and/or 139) are disposed. Further, the sensor module (113) includes a flexible, sticky, silicone pad (310). The housing of the sensor module (113) can be temporally attached to a sticky side of the pad (310), or permanently secured to the pad (310) (e.g., via glue, or fasteners). The back side (301) of the pad (310) facing away from the housing of the sensor module (113) is sticky and can be used to attach the sensor module (113) to the upper arm (101) of the user (or another part of the user).

The sticky pad (310) can be replaceable. For example, when the pad (310) loses its stickiness over time, the pad (310) can be removed from the housing of the sensor module (113); and another sticky pad can be attached to the sensor module (113) to replace the pad (310).

The sticky part of the sensor module (113) can be detachable from the sensor module (113) and/or the pad (310). For example, the sticky part and/or the stick pad (310) can be removably located/mounted on a platform, surface, or mounting structure of the sensor module (113) and/or the housing of the sensor module (113); and a user can optionally choose to use the sticky pad (310) for attaching the sensor module (113) to the upper arm (101) (e.g., in a way as illustrated in FIG. 7) or choose to use an elastic armband (111) for attaching the sensor module (113) to the upper arm (101) (e.g., in a way as illustrated in FIG. 1), or choose to use both the sticky pad (310) and the elastic armband (111) for attaching the sensor module (113) to the upper arm (101).

In at least some embodiments, a system is configured to track both the orientations of parts of the user (e.g., head, arms, hands, torso, legs) but also the biological responses of the user to control the operation of a computing device (e.g., 141).

For example, the calibration and calculation of the orientations of parts of the user can be performed using inertial measurement units, as discussed in U.S. patent application Ser. No. 15/787,555, filed Oct. 18, 2017, issued as U.S. Pat. No. 10,379,613 on Aug. 13, 2019, and entitled “Tracking Arm Movements to Generate Inputs for Computer Systems,” U.S. patent application Ser. No. 15/492,915, filed Apr. 20, 2017, published as U.S. Pat. App. Pub. No. 2017/0308165 on Oct. 26, 2017, and entitled “Devices for Controlling Computers based on Motions and Positions of Hands,” U.S. patent application Ser. No. 15/817,646, filed Nov. 20, 2017, published as U.S. Pat. App. Pub. No. 2018/0313867 on Nov. 1, 2018, and entitled “Calibration of Inertial Measurement Units Attached to Arms of a User to Generate Inputs for Computer Systems,” U.S. patent application Ser. No. 15/813,813, filed Nov. 15, 2017, published as U.S. Pat. App. Pub. No. 2018/0335834 on Nov. 22, 2018, and entitled “Tracking Torso Orientation to Generate Inputs for Computer Systems,” U.S. patent application Ser. No. 15/847,669, filed Dec. 19, 2017, published as U.S. Pat. App. Pub. No. 2019/0187784 on Jun. 20, 2019, and entitled “Calibration of Inertial Measurement Units Attached to Arms of a User and to a Head Mounted Device,” U.S. patent application Ser. No. 15/868,745, filed Jan. 11, 2018, published as U.S. Pat. App. Pub. No. 2019/0212359 on Jul. 11, 2019, and entitled “Correction of Accumulated Errors in Inertial Measurement Units Attached to a User,” U.S. patent application Ser. No. 15/864,860, filed Jan. 8, 2018, published as U.S. Pat. App. Pub. No. 2019/0212807 on Jul. 11, 2019, and entitled “Tracking Torso Leaning to Generate Inputs for Computer Systems,” U.S. patent application Ser. No. 15/973,137, filed May 7, 2018, and entitled “Tracking User Movements to Control a Skeleton Model in a Computer System,” U.S. patent application Ser. No. 15/996,389, filed Jun. 1, 2018, issued as U.S. Pat. No. 10,416,755 on Sep. 17, 2019, and entitled “Motion Predictions of Overlapping Kinematic Chains of a Skeleton Model used to Control a Computer System,” U.S. patent application Ser. No. 16/044,984, filed Jul. 25, 2018, and entitled “Calibration of Measurement Units in Alignment with a Skeleton Model to Control a Computer System,” U.S. patent application Ser. No. 16/375,108, filed Apr. 4, 2019, and entitled “Kinematic Chain Motion Predictions using Results from Multiple Approaches Combined via an Artificial Neural Network,” U.S. patent application Ser. No. 16/534,674, filed Aug. 7, 2019, and entitled “Calibration of Multiple Sensor Modules Related to an Orientation of a User of the Sensor Modules,” the entire disclosures of which applications are hereby incorporated herein by reference.

Further, the sensor system can be configured to track the physical indicators and biometric parameters of user and the environment parameters.

For example, a heart rate sensor can be used to measure the heart rate in beats per minute and/or detect the pulse waves of the user. The heart rate sensor can be an optical heart rate sensor, or a photoplethysmography sensor.

For example, a thermometer can be configured to measure the temperature of the user.

For example, a manometer can be configured to measure the blood pressure of the user.

For example, a galvanic skin sensor can be configured to measure the skin resistance, skin conductance and stress level of the user (e.g., to detect sweating and stress level).

For example, an electromyography sensor can be configured to measure the electrical activity of muscles of the user.

For example, a CGM (continuous glucose monitoring) sensor can be configured to monitor the glucose level of the user.

Other types of sensors can be used to measure biometric parameters, physical parameters, and/or biological responses of the user.

Environmental conditions surrounding the user can also be measured to calibrate and/or interpret the biological responses of the user measured using biological response sensors.

For example, a thermometer can be configured to measure the ambient temperature surrounding the user.

For example, a barometric sensor can be configured to measure the air pressure surrounding the user.

For example, a pressure sensor can be configured to measure the pressure of gases or liquids surrounding the user.

For example, a humidity/hygrometer sensor can be configured to measure moisture level in the air surrounding the user.

For example, an altimeter can be configured to measure the altitude of the user above a fixed level.

For example, a capacitive sensor/proximity sensor can be configured to detect and measure proximity of objects near the user. In an embodiment, the capacitive sensor/proximity sensor is configured to activate the computing device in response to sensing that the user is wearing the device.

For example, an optical sensor can be configured to detect and measure proximity of objects near the user.

For example, a UV-light sensor can be configured to measure global UV radiance surrounding the user.

For example, a GPS (global positioning system) receiver can be configured to detect the current location of the user.

Other types of sensors can be used to measure the environmental parameters relevant to the biometric, physical, and/or biological parameters of the user.

In general, biological response sensors can include sensors directly measuring the responses of the user and the sensors measuring the relevant environmental parameters, such as the examples of sensors discussed above.

Some of the biological response sensors can be integrated within the housing of sensor modules (e.g., 113, 115) having IMUs. Some of the biological response sensors can be disposed within separate housing that has not IMUs. In some implementations, a collection of sensors measuring the relevant environmental parameters can be configured in a separate module that services as a base unit to gather sensor data from other sensor modules.

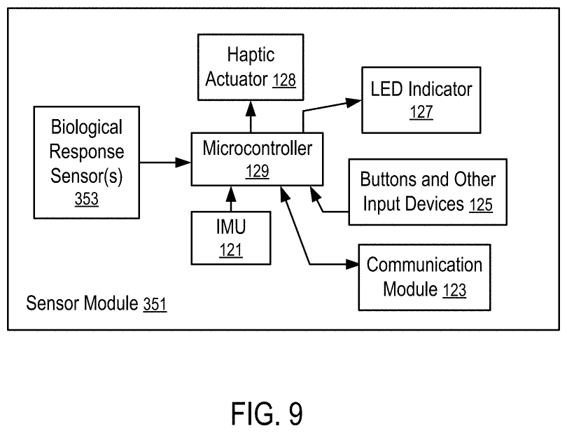

FIG. 9 shows a sensor module (351) having one or more biological response sensors (353) and an inertial measurement unit (121) according to one embodiment. For example, the sensor module (351) can be a handheld module (115), or an arm module (113).

Similar to the handheld module (115) and/or the arm module (113) of FIG. 2, the sensor module (113) of FIG. 9 can have electronic components, such as an LED indicator (127), a haptic actuator (128), a microcontroller (129), a communication module (123), and/or the buttons and other input devices (125). In an embodiment, the LED indicator (127) can include an LED display and/or one or more LED lights.

In FIG. 9, the sensor module (351) further includes a biological response sensor (353) that is configured to measure the biological response parameters of the user, such as heat rate, temperature, blood pressure, sweating/stress, electrical activity of muscles, or glucose level, or any combination thereof. In an embodiment, the sensor module (351) can be used to signal a personal emergency or other security concern. For example, the sensor module (351) can have a special physical or virtual button, or a special combination of gestures should be made by a user on a touchpad surface of the device to send an emergency signal to the main computing device (141).

Optionally, the biological response sensor(s) (353) of the sensor module can further measure at least some environmental parameters that are relevant to the biological responses of the user, such as ambient temperature, air pressure, pressure of gases or liquids, moisture level, altitude, presence of nearby objects, UV light level, or geographical local, or any combination thereof.

In some embodiments, the sensor module (351) has the biological response sensor(s) (353) but no IMU (121).

In general, a computing device (141) as illustrated in FIG. 2 can receive inputs from multiple modules (e.g., 115, 113, 351). Some of the modules (e.g., 115, 113, 351) can have biological response sensor(s) (353) but no IMU; some of the modules (e.g., 115, 113, 351) can have IMUs (e.g., 121, 131) but no biological response sensor(s) (353); and other modules (e.g., 115, 113, 351) can have both IMUs (e.g., 121, 131) and biological response sensor(s) (353). Different sensor modules configured for attaching to different parts of the user can have different sets of biological response sensor(s) (353).

The computing device (141) can be configured to combine the input data from the sensor modules (e.g., 113, 115, 351), including motion data and biological responses, to generate inputs to control the application (147).

For example, the computing device (141) can be an augmented reality (AR)/virtual reality (VR) helmet or glass, a smartphone, a personal computer (PC), etc.

The measured data about biometric, physical, biological parameters can be analyzed in the computing device (141) to control the application (147) (e.g., to adjust output, activate/deactivate tool, manipulate the user interface elements).

For example, in response to an increased heart rate and increased walk pace detected via the biological response sensor(s) (353), the computing device (141) can assume that sport activities have started and thus, adapt the user interface automatically to display information about current type of sport activity. For example, a VR/AR display associated with jogging can present information about distance, average pace, duration, etc.

For example, in response to a humidity sensor detects increased moisture, and pressure sensor indicates that user is in the water, a VR/AR display associated with swimming can be presented.

For example, during a physical training exercise, a combination of body tracking and biological response sensing of the user can be used in the computing device (141) to detect the correctness of exercise and present demonstrations on a head mounted display, a smartphone, or a TV screen.

For example, in response to detecting that measured biological or physical parameters reaching a threshold value (for increased/decreased heart rate, temperature, glycose level, or blood pressure), the computer device (141) can present a window with a warning message and/or instructions to the user. Further, haptic feedback can be provided via the haptic actuator(s) (e.g., 128, 138).

For example, using data received from biological response sensors and IMU sensors, the computing device (141) can track the activities of the user within a day.

For example, based on the received data from a GPS receiver, the computing device (141) can determined the current geographical location of the user (e.g., at home, on the street, at work, etc.) and adjusts the user interface based on the current location of the user.

For example, based on the received data about environment, the computing device (141) can adjust the current weather report provided on the user interface displayed.

For example, the computing device (141) can be configured to change graphical user interface (GUI) design based on the UV-light measurements.

For example, in response to a proximity sensor (or GPS tracking device) detecting the presence of other users with similar devices nearby the user, the computing device (141) can be configured to automatically display a window with invitation to activate multiplayer mode to further interaction with other users.

In general, the biological response sensor(s) (353) can be attached to anywhere on the body of the user. Multiple sensor modules (e.g., 351) each having one or more biological response sensors (353) (but may or may not have an IMU) can be attached to multiple places (e.g., on a leg, on the back) of the body of the user at the same time to generate biological response inputs for the computing device (141). Multiple sensor modules (e.g., 351) can be used simultaneously to generate more accurate positional and biological response data to improve performance. For example, when the computing device (141) can be configured to generate virtual reality (VR) or augmented reality (AR) display during physical exercise, athletic practice/performance/training; and the positional and biological response data generated by the sensor modules (e.g., 115, 113, 351) can be used to enhance the VR/AR display for improving the performance of the user in the activities. The sensor modules (e.g., 115, 113, 351) can be used and placed anywhere in the body to provide more biological and/or positional feedback. Further, sensor modules configured to measure certain environmental parameters can be placed at locations close to the user without being attached to the body of the user.

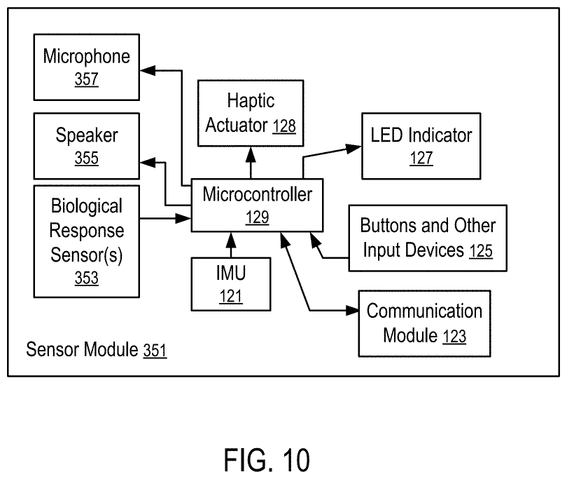

FIG. 10 shows another sensor module having biological response sensors, an inertial measurement unit, and audio input/output devices according to one embodiment.

The sensor module (351) of FIG. 10 has components (121 to 129 and 353) that are similar to corresponding components (121 to 129 and 353) in the sensor module (351) of FIG. 9. In addition, the sensor module (351) of FIG. 10 has audio input/output devices, such as a speaker (355) and a microphone (357).

For example, the sensor module (351) of FIG. 10 can receive input audio data generated by a user (e.g., input voice commands) using a microphone (357) mounted on the sensor module (351). The computing device (141) can collect the audio data (e.g., voice commands) through the microphone (357), the microcontroller (129) and the communication device (123). The computing device (141) can be an augmented reality (AR)/virtual reality (VR) helmets or glasses, smartphones, PC, etc.). The computing device (141) can process transferred data and generate output audio data (e.g., output voice prompts), and transfer generated output data to the sensor module (351) via the communication module (123) (e.g., a wireless transceiver). The microcontroller (129) plays to a user the output audio data received from the computing device (141) using the speaker (355) located on sensor module (351).

Alternatively, or in additional to the microphone (347) and the speaker (355) illustrated in FIG. 10, the sensor module (351) can include one or more other additional input/output elements, such as: a touchpad to identify the motion or position of a user's finger; an optical sensor to measure proximity of nearby objects; a piezoelectrical sensor to measure changes in pressure, acceleration, temperature, strain, or force; a fingerprint scanner to determine the identity of the user; a camera to provide optical input data to the main computing device (141); one or more LED (Light-Emitting Diode) indicators to provide optical output data; one or more LED displays to provide output data received from the main computing device (141); a separate magnetic sensor (not as a part of IMU sensors (121) but a part of the sensor module (351)) to detect changes and disturbances in a magnetic field like flux, strength and direction; a motion detector system based on passive infrared (PIR) sensors/ultrasonic transducer or it's combination with other sensors to detect moving objects; a SIM (subscriber identification module) card slot, which can support 5G connection; a Near Field Communication (NFC) marker to exchange the information with other devices; or one or more haptic actuator(s) to translate tactile output data received from the main computing device (141); or any combination therein.

For example, the sensor module (351) can be configured to receive input data generated by a user (e.g., swipe-up, swipe-down, fingerprint) using the additional input/output elements discussed above and collect the information through the microcontroller (129). The sensor module (351) can transfer the collected data to the main computing device (141) (e.g., AR/VR helmets or glasses, smartphones, PC, etc.). The main computing device (141) can process transferred data and generate output data (e.g., output voice commands, haptic feedback, image/video information), and transfer the generated output data via wireless transceiver to one or more additional output elements configured in the sensor module (351) for presentation to the user of the sensor module (351). Using the additional input/output elements, the system can provide output information and tools viewable on the user interface in an AR space or on LED display(s) added to the sensor module (351).

In an embodiment, the sensor module (351) can be used to signal a personal emergency or other security concern. For example, the sensor module (351) can have a special physical or virtual button, or a special combination of gestures should be made by a user on a touchpad surface of the device to send an emergency signal to the main computing device (141).

For example, using 5G connection or/and NFC markers, the sensor module (351) can quickly connect to other devices nearby (e.g., connecting to the main computing device (141) via Bluetooth/WiFi without requesting of special actions from the user).

For example, using 5G connection or/and NFC markers, or/and a motion detector system, the sensor module (351) can detect other devices nearby (i.e., devices which support Internet-of-Things (IoT) system). In combination with other additional components, the system can adapt current UI tools on a VR/AR space, or on the LED display of the main computing device (141), or on the LED display of the sensor module (351). For example, if an IoT device is located more than 2 m from a user, main information about IoT device can be displayed (e.g., battery level, current status, etc.). If IoT device is located closer than 2 m from a user, additional information about IoT device can be displayed (e.g., tools to control IoT device, to change its operating status, to turn on or off sleep mode, to issue a command, etc.).

For example, interaction with the alarm systems of other devices can be implemented using fingerprint/voice scanner (e.g., to lock or unlock devices).

For example, using a touchpad or/and a piezoelectric sensor, and/or optical sensor, the system can recognize simple gestures, such as swipe, click, tap, squeeze, etc., to activate certain commands. For example, combination of taps or squeeze can open or close active interface window; and swipe-up or swipe-down can hidden active interface window.

For example, combination of LED display(s), LED indicators and haptic actuators can provide output data to a user (e.g., missed call, low-level battery, information about devices detected nearby).

For example, a combination of biometric sensors and the additional input/output elements can provide more accurate information about the current status of the sensor module (351) and the biometric parameters of a user. For example, using data received from GPS receiver and magnetic sensor, the sensor module (351) can guide a user, using haptic actuators, speaker, LED display and LED indicators, along the selected route.

The present disclosure includes methods and apparatuses which perform these methods, including data processing systems which perform these methods, and computer readable media containing instructions which when executed on data processing systems cause the systems to perform these methods.

For example, the computing device (141), the arm module (113) and/or the handheld module (115) can be implemented using one or more data processing systems.

A typical data processing system may include includes an inter-connect (e.g., bus and system core logic), which interconnects a microprocessor(s) and memory. The microprocessor is typically coupled to cache memory.

The inter-connect interconnects the microprocessor(s) and the memory together and also interconnects them to input/output (I/O) device(s) via I/O controller(s). I/O devices may include a display device and/or peripheral devices, such as mice, keyboards, modems, network interfaces, printers, scanners, video cameras and other devices known in the art. In one embodiment, when the data processing system is a server system, some of the I/O devices, such as printers, scanners, mice, and/or keyboards, are optional.

The inter-connect can include one or more buses connected to one another through various bridges, controllers and/or adapters. In one embodiment the I/O controllers include a USB (Universal Serial Bus) adapter for controlling USB peripherals, and/or an IEEE-1394 bus adapter for controlling IEEE-1394 peripherals.

The memory may include one or more of: ROM (Read Only Memory), volatile RAM (Random Access Memory), and non-volatile memory, such as hard drive, flash memory, etc.

Volatile RAM is typically implemented as dynamic RAM (DRAM) which requires power continually in order to refresh or maintain the data in the memory. Non-volatile memory is typically a magnetic hard drive, a magnetic optical drive, an optical drive (e.g., a DVD RAM), or other type of memory system which maintains data even after power is removed from the system. The non-volatile memory may also be a random access memory.

The non-volatile memory can be a local device coupled directly to the rest of the components in the data processing system. A non-volatile memory that is remote from the system, such as a network storage device coupled to the data processing system through a network interface such as a modem or Ethernet interface, can also be used.

In the present disclosure, some functions and operations are described as being performed by or caused by software code to simplify description. However, such expressions are also used to specify that the functions result from execution of the code/instructions by a processor, such as a microprocessor.

Alternatively, or in combination, the functions and operations as described here can be implemented using special purpose circuitry, with or without software instructions, such as using Application-Specific Integrated Circuit (ASIC) or Field-Programmable Gate Array (FPGA). Embodiments can be implemented using hardwired circuitry without software instructions, or in combination with software instructions. Thus, the techniques are limited neither to any specific combination of hardware circuitry and software, nor to any particular source for the instructions executed by the data processing system.

While one embodiment can be implemented in fully functioning computers and computer systems, various embodiments are capable of being distributed as a computing product in a variety of forms and are capable of being applied regardless of the particular type of machine or computer-readable media used to actually effect the distribution.

At least some aspects disclosed can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

Routines executed to implement the embodiments may be implemented as part of an operating system or a specific application, component, program, object, module or sequence of instructions referred to as “computer programs.” The computer programs typically include one or more instructions set at various times in various memory and storage devices in a computer, and that, when read and executed by one or more processors in a computer, cause the computer to perform operations necessary to execute elements involving the various aspects.

A machine readable medium can be used to store software and data which when executed by a data processing system causes the system to perform various methods. The executable software and data may be stored in various places including for example ROM, volatile RAM, non-volatile memory and/or cache. Portions of this software and/or data may be stored in any one of these storage devices. Further, the data and instructions can be obtained from centralized servers or peer to peer networks. Different portions of the data and instructions can be obtained from different centralized servers and/or peer to peer networks at different times and in different communication sessions or in a same communication session. The data and instructions can be obtained in entirety prior to the execution of the applications. Alternatively, portions of the data and instructions can be obtained dynamically, just in time, when needed for execution. Thus, it is not required that the data and instructions be on a machine readable medium in entirety at a particular instance of time.

Examples of computer-readable media include but are not limited to non-transitory, recordable and non-recordable type media such as volatile and non-volatile memory devices, read only memory (ROM), random access memory (RAM), flash memory devices, floppy and other removable disks, magnetic disk storage media, optical storage media (e.g., Compact Disk Read-Only Memory (CD ROM), Digital Versatile Disks (DVDs), etc.), among others. The computer-readable media may store the instructions.

The instructions may also be embodied in digital and analog communication links for electrical, optical, acoustical or other forms of propagated signals, such as carrier waves, infrared signals, digital signals, etc. However, propagated signals, such as carrier waves, infrared signals, digital signals, etc. are not tangible machine readable medium and are not configured to store instructions.

In general, a machine readable medium includes any mechanism that provides (i.e., stores and/or transmits) information in a form accessible by a machine (e.g., a computer, network device, personal digital assistant, manufacturing tool, any device with a set of one or more processors, etc.).

In various embodiments, hardwired circuitry may be used in combination with software instructions to implement the techniques. Thus, the techniques are neither limited to any specific combination of hardware circuitry and software nor to any particular source for the instructions executed by the data processing system.

In the foregoing specification, the disclosure has been described with reference to specific exemplary embodiments thereof. It will be evident that various modifications may be made thereto without departing from the broader spirit and scope as set forth in the following claims. The specification and drawings are, accordingly, to be regarded in an illustrative sense rather than a restrictive sense.

What is claimed is:

1. A system, comprising:
a plurality of sensor modules configured to be attached to a user, at least a first of the plurality of sensor modules having an inertial measurement unit, and at least a second of the plurality of sensor modules having one or more biological response sensors having a biological sensor module; and
a computing device in communication with at least one of the plurality of sensor modules, the computing device configured to control an application based on motion data of the user measured by inertial measurement units in the plurality of sensor modules and biological responses determined by the biological sensor module.

2. The system of claim 1, wherein each of the plurality of sensor modules has a communication module to transmit measurements to the computing device.

3. The system of claim 2, wherein the inertial measurement unit includes a micro-electromechanical system (MEMS) gyroscope.

4. The system of claim 3, wherein the one or more biological response sensors include:
a heart rate sensor;
a thermometer;
a manometer;
a galvanic skin sensor;
an electromyography sensor;
a continuous glucose monitoring sensor;
a barometric sensor;
a pressure sensor;
a humidity sensor;
a hygrometer;
an altimeter;
a capacitive proximity sensor;
an optical proximity sensor;
a UV-light sensor;
a global positioning system (GPS) receiver;
a fingerprint scanner; or
any combination thereof.

5. The system of claim 4, comprising a display in communication with the computing device, wherein, in response to a combination of motion measurements meeting a first predetermined criterion and biological responses meeting a second predetermined criterion, the computing device is configured to:
activate a virtual reality application;
activate an augmented reality application;
display a window with a warning message;
display a window with instructions;
change a user interface design; or
any combination thereof.

6. The system of claim 5, wherein at least one of the plurality of sensor modules has a microphone and configured to transmit audio data from the microphone to the computing device; and at least one of the plurality of sensor modules has a speaker and configured to transmit audio data from the computing device to the speaker.

7. The system of claim 4, wherein in response to a combination of motion measurements meeting a first predetermined criterion and biological responses meeting a second predetermined criterion, the computing device is configured to emit an indication of a personal emergency.

8. The system of claim 1, wherein the first of the plurality of sensor modules includes a communication module and first of the plurality of sensor modules is in electronic communication with the plurality of sensor modules and the computing device.

9. The system of claim 1, wherein the computing device is configured to:
receive motion data from the first of the plurality of sensor modules identifying an orientation of an upper arm of the user;
receive motion data from a second of the plurality of sensor modules identifying an orientation of a hand of the user; and
calculate an orientation of a forearm of the user connecting the hand and the upper arm, wherein the orientation is calculated without a sensor module being present on the forearm.

10. The system of claim 1, wherein the first of the plurality of sensor modules comprises one or more biological response sensors having a biological sensor module.

11. The system of claim 1, wherein the second of the plurality of sensor modules comprises an inertial measurement unit.

12. The system of claim 1, further comprising a proximity sensor and a near field communication marker, wherein in response to detecting presence of a nearby computing device, the computing device is configured to display a window with an invitation to active a multiplayer mode and interact with the nearby computing device via the near field communication marker.

13. The system of claim 1, further comprising a plurality of biological response sensors disposed at different locations of the user.

14. The system of claim 1, wherein the plurality of sensor modules comprises two sensor modules, each of the two sensor modules comprising an inertial measurement unit and one or more biological sensors.

15. A method, comprising:
receiving, by a computing device, motion data from an arm module identifying an orientation of an upper arm of a user;
receiving, by the computing device, motion data from a handheld module identifying an orientation of a hand of the user;
receiving, by the computing device, biological responses from a biological sensor module;
calculating, by the computing device, an orientation of a forearm of the user connecting the hand and the upper arm, wherein the orientation is calculated without a sensor module being present on the forearm; and
controlling an application based on the calculated orientation of the forearm of the user, the received motion data from the arm module, the received motion data from the handheld module, and the biological responses.

16. The method of claim 15, wherein the biological responses are received from one or more biological sensors including:
a heart rate sensor;
a thermometer;
a manometer;
a galvanic skin sensor;
an electromyography sensor;
a continuous glucose monitoring sensor;
a barometric sensor;
a pressure sensor;
a humidity sensor;
a hygrometer;
an altimeter;
a capacitive proximity sensor;
an optical proximity sensor;
a UV-light sensor;
a global positioning system (GPS) receiver;
a fingerprint scanner; or
any combination thereof.

17. The method of claim 15, comprising generating an emergency alert in response to the biological responses.

18. The method of claim 15, comprising, wherein, in response to a combination of motion measurements meeting a first predetermined criterion and biological responses meeting a second predetermined criterion:
activating a virtual reality application;
activating an augmented reality application;
displaying a window with a warning message;
displaying a window with instructions;
changing a user interface design; or
any combination thereof.

19. The method of claim 15, comprising detecting presence of a nearby computing device, and displaying a window with an invitation to active a multiplayer mode and interact with the nearby computing device.

20. The method of claim 15, wherein controlling the application based on the calculated orientation of the forearm of the user, the received motion data from the arm module, the received motion data from the handheld module, and the biological responses comprises, in response to an increased heart rate, and an increased walking pace, displaying a window with instructions associated with a jogging activity.
